(19) United States
(12) Patent Application Publication
(10) Pub. No.: US 2016/0034636 A1
(43) Pub. Date: Feb. 4, 2016

(54) OLIGONUCLEOTIDE PROBES FOR SPECIFIC IDENTIFICATION OF NOROVIRUSES AND OTHER PATHOGENS

(71) Applicant: The United States of America, as represented by the Secretary of Agriculture, Washington, DC (US)

(72) Inventors: Beatriz Quiñones, San Pablo, CA (US); Bertram G Lee, San Francisco, CA (US)

(21) Appl. No.: 14/724,736

(22) Filed: May 28, 2015

Related U.S. Application Data
(60) Provisional application No. 62/030,577, filed on Jul. 29, 2014.

(51) Int. Cl. G06F 19/20; G06F 19/22; C12Q 1/70; C40B 30/02

(52) U.S. Cl. CPC G06F 19/20; C40B 30/02; G06F 19/22; C12Q 1/701; C12Q 2600/158

(57) ABSTRACT

Embodiments of the present disclosure relate to the identification of norovirus genotypes using type specific probes. Other embodiments of the present disclosure relate to the identification of distinct types of organisms using type specific probes.

### REFERENCE TO RELATED APPLICATIONS

This application claims priority to U.S. Ser. No. 62/030,577, filed Jul. 29, 2014 which is incorporated by reference herein.

### FIELD OF THE INVENTION

The invention relates to methods for sensitive detection of individual norovirus genotypes in a sample having one or more norovirus strains.

### REFERENCE TO A COMPUTER PROGRAM LISTING APPENDIX

This application includes a computer program listing appendix. The computer program listing appendix is submitted herewith in accordance with 37 C.F.R. 1.96(c), and the material comprising the computer program listing appendix is incorporated herein by reference in its entirety.

### BACKGROUND OF THE INVENTION

Noroviruses are a genetically diverse group of single-stranded RNA, non-enveloped viruses in the Caliciviridae family. Noroviruses are a leading cause of foodborne illness, and are responsible for over half of the foodborne illness in the world.

Foodborne illness caused by noroviruses is in most cases is usually moderate and self-limiting and only lasts about 1-3 days. However, the acute gastroenteritis caused by norovirus is unpleasant and widespread. Indeed, in the United States, approximately 5.5 million illnesses attributable to norovirus are estimated to occur annually. Thus, these foodborne viruses cause about two billion dollars in health care expenses and lost productivity in the United States alone (see e.g., Scallan et al., (2011) Emerg Infect Dis 17: 7-15). Furthermore, the severity of disease infection can be especially virulent in the young, elderly, and immunocompromised persons who are at risk for complications caused by severe vomiting and diarrhea. Thus, these foodborne viruses also cause about 150 deaths per year in the United States alone.

Noroviruses are genetically classified into 5 genogroups, GI-GV on the basis of similarity across highly conserved regions of the genome (see e.g., Zheng, D. P. et al. (2006) Virology 346:312-323). Within the different genogroups multiple subtypes/genotypes are known. For example, in genogroup I, at least 8 genotypes are currently recognized and in genogroup II, at least 19 genotypes are recognized. Genogroups I and II cause virtually all of the human disease. In particular, noroviruses of the Genogroup II genotype 4 (GII.4) are the predominant circulating strains and are responsible for more than 85% of all outbreaks.

Typical methods for norovirus detection and identification include real-time reverse transcription-polymerase chain reaction (RT-PCR) assays, DNA sequencing of conventional RT-PCR products and enzyme immunoassay (EIA). However, none of the methods currently in use are able distinguish specific genotypes but only specific genogroups.

New epidemic variants of GII.4 emerge every few years. New variants may be associated with different and unexpected characteristics which may lead to pandemics of acute gastroenteritis caused by some variants but not others.

Therefore, because norovirus is the leading cause of human gastroenteritis, and because different genotypes can cause different degrees of disease severity and to identify sources of contamination leading to foodborne outbreaks, there is a need in the art for improved methods that permit sensitive, simultaneous detection of different norovirus genotypes. Fortunately, as will be clear from the following disclosure, the present invention provides for these and other needs.

### SUMMARY OF THE INVENTION

One embodiment of the disclosure provides a method designing a probe specific for a target subtype, wherein the probe specific for the target subtype distinguishes between the target subtype and all known non-target subtypes, the method comprising: (i) providing in silico genomic data comprising an alignment of nucleotide sequences that comprise the target subtype; (ii) sequentially scanning the in silico genomic data comprising the alignment of nucleotide sequences that comprise the target subtype, using a select comparison window, to determine a region of low variability between the nucleotide sequences that comprise the target subtype, thereby identifying one or more potential probes, each of which are a length in nucleotides equal to the select comparison window;

(iii) providing an in silico alignment of nucleotide sequences of each of the known non-target subtypes; (iv) testing the one or more potential probes against the in silico alignment of nucleotide sequences of each of the non-target subtypes to determine if any of the potential probes have high variability with the nucleotide sequences of the in silico alignment of nucleotide sequences of each of the non-target subtypes, thereby identifying one or more specific probes; (v) selecting the specific probes that show low variability to the in silico nucleotide sequences of the target subtype and high variability to in silico alignment of nucleotide sequences of each of the non-target subtypes, thereby designing the specific probes for the target subtype. In one exemplary embodiment, the low variability is at least 90% sequence identity and wherein the low variability is less than 79%. In another exemplary embodiment, the target subtype is a member selected from the group consisting of a norovirus genotype, a hepatitis A virus, a serovar of Salmonella enterica and a strain of Escherichia coli.

Another embodiment of the disclosure provides a method for designing a probe specific for a target norovirus genotype, wherein the probe specific for the target norovirus genotype distinguishes between the target norovirus genotype and all known non-target norovirus genotypes, the method comprising: (i) providing in silico genomic data comprising an alignment of nucleotide sequences from the target norovirus genotype; (ii) sequentially scanning the in silico genomic data comprising the alignment of nucleotide sequences from the target norovirus genotype using a select comparison window to determine a region of low variability between the nucleotide sequences of the target norovirus genotype, thereby identifying one or more potential probes, each of which are a length in nucleotides equal to the select comparison window; (iii) providing an in silico alignment of nucleotide sequences of each of the known non-target norovirus genotypes; (iv) testing the one or more potential probes against the in silico alignment of nucleotide sequences of each of the non-target norovirus genotypes to determine if any of the potential probes have high variability with the nucleotide sequences of the in silico alignment of nucleotide sequences of each of the non-target norovirus genotypes, thereby identifying specific probes; (v) selecting the specific probes that show low variability to the alignment of nucleotide sequences from the target norovirus genotype and high variability to in silico alignment of nucleotide sequences of each of the non-target norovirus genotypes, thereby designing the specific probes for the target norovirus genotype. In one exemplary embodiment, the low variability is at least 90% sequence identity and the high variability is less than 75% sequence identity. In one exemplary embodiment, the select comparison window is between about 20 nucleotides to about 40 nucleotides.

Another embodiment of the disclosure provides a non-transitory computer readable medium containing computer instructions stored therein for causing a computer processor to perform a method for designing a probe specific for a target norovirus wherein the method for designing the probe specific for a target norovirus strain comprises: (i) providing in silico genomic data comprising an alignment of nucleotide sequences from the target norovirus genotype; (ii) sequentially scanning the in silico genomic data comprising the alignment of nucleotide sequences from the target norovirus genotype using a select comparison window to determine a region of low variability between the nucleotide sequences of the target norovirus genotype, thereby identifying one or more potential probes, each of which are a length in nucleotides equal to the select comparison window; (iii) providing an in silico alignment of nucleotide sequences of each of the known non-target norovirus genotypes; (iv) testing the one or more potential probes against the in silico alignment of nucleotide sequences of each of the non-target norovirus genotypes to determine if any of the potential probes have high variability with the nucleotide sequences of the in silico alignment of nucleotide sequences of each of the non-target norovirus genotypes, thereby identifying specific probes; (v) selecting the specific probes that show low variability to the alignment of nucleotide sequences from the target norovirus genotype and high variability to in silico alignment of nucleotide sequences of each of the non-target norovirus genotypes, thereby designing the specific probes for the target norovirus genotype.

Another embodiment of the disclosure provides a method for determining if a target norovirus genotype is present in a sample comprising a population of noroviruses and other unrelated organisms, the method comprising: (i) designing a probe specific for the target norovirus wherein designing the probe specific for the target norovirus comprises: (a) providing in silico genomic data comprising an alignment of nucleotide sequences from the target norovirus genotype; (b) sequentially scanning the in silico genomic data comprising the alignment of nucleotide sequences from the target norovirus genotype using a select comparison window to determine a region of low variability between the nucleotide sequences of the target norovirus genotype, thereby identifying one or more potential probes, each of which are a length in nucleotides equal to the select comparison window; (c) providing an in silico alignment of nucleotide sequences of each of the known non-target norovirus genotypes; (d) testing the one or more potential probes against the in silico alignment of nucleotide sequences of each of the non-target norovirus genotypes to determine if any of the potential probes have high variability with the nucleotide sequences of the in silico alignment of nucleotide sequences of each of the non-target norovirus genotypes, thereby identifying specific probes; (e) selecting the specific probes that show low variability to the alignment of nucleotide sequences from the target norovirus genotype and high variability to in silico alignment of nucleotide sequences of each of the non-target norovirus genotypes, thereby designing the specific probes for the target norovirus genotype; (ii) attaching the specific probes for the target norovirus to a microarray slide; (iii) preparing labeled cDNA from a sample comprising target norovirus nucleotide sequences and non-target norovirus nucleotide sequences so that the labeled cDNA comprises labeled target norovirus cDNA and labeled non-target norovirus cDNA; (iv) hybridizing the labeled cDNA comprising the labeled target norovirus cDNA and labeled non-target norovirus cDNA to the microarray slide to which the specific probes for the target norovirus genotype(s) are attached; (v) detecting a hybridization signal when the labeled target norovirus cDNA specifically hybridizes to the specific probe for the target norovirus attached to the microarray slide, thereby determining if a target norovirus is present in a sample comprising a population of noroviruses and other unrelated organisms.

Other features, objects and advantages of the invention will be apparent from the detailed description which follows.

### BRIEF DESCRIPTION OF THE DRAWINGS

FIG. 1 illustrates exemplary amplified regions for genotyping in the genomic RNA of norovirus.

FIG. 2 illustrates how one embodiment of the invention detects target DNA in a colorimetric microarray.

FIG. 3 shows alignments demonstrating the process of how probes are generated.

FIG. 4 is a graph showing the specificity of selected probes targeting region C to different norovirus strains, with a sample layout on the top right corner.

FIG. 5 is a graph showing the sensitivity of select probes targeting region C to different norovirus strains.

FIG. 6 is a graph showing the sensitivity of selected probes to different concentrations of norovirus GII.4 cRNA.

FIG. 7 is a flowchart illustrating method steps programmed in the computer software.

### DETAILED DESCRIPTION OF THE INVENTION

### Definitions

The term “virus” as used herein, refers to sub-microscopic infectious agents that are typically unable to grow or reproduce outside a host cell. Typically a viral particle, or virion, comprises genetic material e.g., DNA or RNA, within a protective protein coat called a capsid. Viruses infect all cellular life forms and are grouped into animal, plant and bacterial types, according to the type of host infected.

The term “norovirus” as used herein, refers to genetically diverse single-stranded RNA, non-enveloped viruses in the Caliciviridae family. Noroviruses are classified into five “genogroups” on the basis of genetic similarity across highly conserved regions of the genome (see e.g., Zheng, Du Ping et al (2006) Virology 346(2):312-323).

The term “genogroup” or “norovirus genogroup” as used herein refers generally to noroviruses having between about 45% to about 62% sequence difference of the nucleotide sequence of the major capsid protein (ORF2).

The term “genotype” or “norovirus genotype” as used herein, refers to noroviruses having between about 15% to about 43.8% sequence difference of the nucleotide sequence of the major capsid protein (ORF2).

The term “strain” or “norovirus strain” as used herein, refers to genetically distinct noroviruses with a particular genotype typically having less than 15% sequence difference of the nucleotide sequence of the major capsid protein (ORF2).

The term “target subtype” as used herein, refers to an organism to be identified by hybridization to a specific probe. A “target subtype” is typically one member of a group of subtypes that make up a broader grouping or “type”. For example, within a norovirus genogroup (the broader “type”) there may be more than one genotype (subtype). In an exemplary embodiment, one genotype of the more than one genotypes is selected as the “target norovirus genotype” or “target subtype”. Thus, in an exemplary a “target norovirus genotype” is distinguished from “non-target norovirus genotypes” on the level of genotype. In other exemplary embodiments, a target subtype is a “target hepatitis A virus”. In still other exemplary embodiments, a “target subtype” is a “target Escherichia coli”. In still other exemplary embodiments, a “target subtype” is a “target Salmonella serovar”.

The term “probe specific for a target subtype” as used herein, refers to a nucleotide sequence of a defined length, designed according to the methods disclosed herein, that can be used in hybridization experiments (e.g., DNA microarray analysis) to distinguish a “target subtype” from “non-target subtype” (e.g., a “target norovirus genotype” is distinguished from “non-target norovirus genotype”). Typically, a “probe specific for a target subtype” has at least 90% sequence identity to the target subtype and no more than 75% sequence identity to non-target subtype. In exemplary embodiments, a “probe specific for a target subtype” is a “probe specific for a target norovirus genotype” and the “probe specific for a target norovirus genotype” is used to distinguish/identify the “target norovirus genotype” from “non-target norovirus genotype” in a sample.

Thus, the expression “distinguishes between the target subtype and all known non-target subtypes” as used herein, refers to the ability of a “probe specific for a target subtype” e.g., a “probe specific for a target norovirus genotype”, to confirm the identity of an organism and/or confirm the presence of that organism in a sample in a hybridization analysis e.g., a DNA microarray analysis.

The term “in silico” as used herein refers generally, to processes taking place via computer calculations. In recent years, the DNA sequences of hundreds of organisms have been decoded and stored in databases. Indeed, the amount of information stored in “in silico databases” continues to grow exponentially as more genomes are sequenced and more proteomes are characterized. Thus, a large body of “in silico” genomic information has been obtained and stored and the amount continues to grow. The information stored in an “in silico database” may be analyzed to inter alia compare or align nucleotide sequences of related (or unrelated) organisms. Thus, in an exemplary embodiment, “in silico” nucleotide sequences are “genomic data” comprising a nucleotide sequence from a target organism e.g., a target norovirus, deduced by computer calculations based on a known DNA sequence.

Thus, the expression “in silico genomic data comprising an alignment of nucleotide sequences from the target subtype” as used herein refers to nucleotide sequences generated by a computer based on known or deduced nucleotide sequences of the members of the group that comprises the target subtype. In some exemplary embodiments, “in silico genomic data comprising an alignment of nucleotide sequences from the target subtype” is a single sequence. However, since different variants of genotypes may have the same phenotype, typically, genomic variants having the same phenotype are all members of the same “target subtype”. Thus, in some exemplary embodiments, “in silico genomic data comprising an alignment of nucleotide sequences from the target subtype” comprises an alignment of all the known variants that comprise the target subtype.

Therefore, in an exemplary embodiment, a norovirus genotype that is a “target subtype” comprises more than one strain. In this embodiment, the “in silico genomic data comprising an alignment of nucleotide sequences from the target subtype” comprises an alignment of the strains that comprise the genotype.

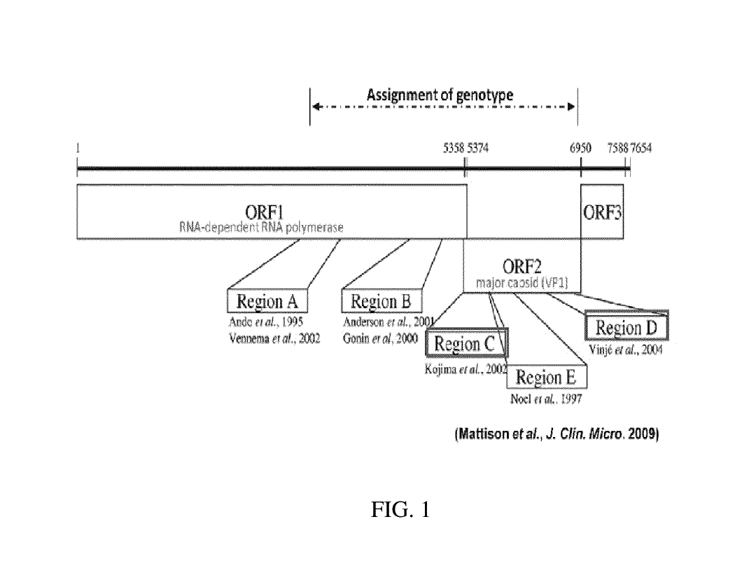

Different regions of the in silico genomic data of a target subtype may be used as analytical segments for preparing specific probes according to the methods disclosed herein. Typically, the region of the in silico genomic data of a target subtype used as analytical segments for preparing specific probes are chosen from those regions of the genome that are evolutionarily conserved e.g., the nucleotide sequence of the major capsid protein of norovirus or a region thereof e.g., the C region of the major capsid protein of norovirus see e.g., FIG. 1, adhesion protein and toxin molecules e.g., Shiga toxin of Escherichia coli, etc. A person having ordinary skill in the art and access to the instant disclosure can readily select one or more analytical segments suitable for their particular analysis.

The expression “in silico alignment of nucleotide sequences of each of the known non-target subtypes” as used herein refers to a theoretical alignment of nucleotide sequences of each of the known non-target subtypes of the type. Typically, the known non-target subtypes of the type are variants that comprise the same broad type as the target subtype, but which are of a different subtype e.g., within the “type” of norovirus genogroup II are the subtypes comprising genotypes 1-19. Thus, if genotype GII.4 is the target norovirus genotype, the remaining genotypes 1-3 and 5-19 comprise the non-target subtypes which are members of the same type (GII genogroup) but are different subtypes than the target norovirus genotype 4.

Typically, an “in silico alignment of nucleotide sequences of each of the known non-target subtypes” is an alignment of a subsequence of the total genomic sequence of the non-target subtypes of the organism/type, typically e.g., a conserved region of the genome e.g., nucleotide sequence of the major capsid protein of a norovirus, that is useful for distinguishing between genetic variants of that organism.

The phrase “select comparison window” as used herein refers to a “comparison window” of a select size e.g. “select comparison window” of 20 nucleotides, a “select comparison window” of 25 nucleotides, a “select comparison window” of 35 nucleotides, etc. In general, a “comparison window”, as used herein, is a segment of any one of the number of contiguous positions selected from the group consisting of from 20 to 600 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

The expression “sequentially scanning” as used herein refers to the analysis of a subsequence of an organism. The subsequence is typically e.g., a conserved region of a genome e.g., nucleotide sequence of the major capsid protein of a norovirus, that is useful for distinguishing between genetic variants of said organism. Analysis by “sequentially scanning” comprises using a select comparison window to locate regions of specified sequence identity within an alignment of a subsequence/analytical segment.

In particular, “sequentially scanning” refers to moving the select comparison window along the aligned genomic data of the subsequence nucleotide by nucleotide (one nucleotide at a time) to locate regions of the aligned genomic data that have a specified level of nucleotide sequence identity. Typically, for a target subtype an alignment of the variants of the target subtype (e.g., the norovirus strains that comprise a particular norovirus genotype represent variants of the subtype which is the norovirus genotype) is sequentially scanned using a select comparison window to identify regions of the alignment where each sequence in the alignment has at least 90% sequence identity to all the other sequences used in the alignment. In some exemplary embodiments, the target subtype is sequentially scanned using a select comparison window to identify regions of the alignment that have at least 91%, at least 92%, at least 93%, at least 94%, at least 95%, at least 96%, at least 97%, at least 98%, at least 99%, at least 100%, sequence identity to all the others.

The expression “potential probe” as used herein, refers to a nucleotide sequence having a length in nucleotides equal to the length of the select comparison window used to find it, wherein the “potential probe” defines a region of the aligned variants of the target subtype where each sequence in the alignment has at least 90% sequence identity to all the other sequences used in the alignment.

The expression “testing the potential probes” as used herein refers to hybridizing in silico potential probes to determine if any of the potential probes have high variability with the nucleotide sequences of the in silico alignment of nucleotide sequences of each of the non-target subtypes. Typically, high variability means that, there is less than about 75% sequence identity between the potential probe and the in silico alignment of nucleotide sequences of each of the non-target subtypes e.g., non-target norovirus genotypes. Potential probes that show high variability to the in silico alignment of nucleotide sequences of each of the non-target subtypes are selected, thereby identifying specific probes.

The expression “specific probe” as used herein refers to a nucleotide sequence with the length of which is equal in length to the select comparison window used to find it, wherein the specific probe defines a region of the aligned variants of the target DNA subtype where each sequence in the alignment of variants has at least 90% sequence identity to all the other target subtype sequences used in the alignment and wherein the defined region also has less that 75% sequence identity to an in silico alignment of nucleotide sequences of each of the non-target subtypes.

The term “computer program” or a “computer readable medium” as used herein refers to non-transitory tangible media containing computer instructions stored therein for causing a computer processor to perform methods disclosed herein. In an exemplary embodiment, a “computer program” as disclosed herein refers to non-transitory tangible media containing computer instructions stored therein for causing a computer processor to perform a method for designing probes that are used to identify an unknown organism e.g., an unknown norovirus. The term “computer program, or computer readable medium” excludes transitory propagating signals per se.

The term “label” as used herein, refers to a composition detectable by spectroscopic, photochemical, biochemical, immunochemical, or chemical means. Exemplary labels include ³²P, fluorescent dyes, electron-dense reagents, enzymes (e.g., as commonly used in an ELISA), biotin, digoxigenin, or haptens and proteins for which antisera or monoclonal antibodies are available.

Thus, the term “labeled nucleic acid probe or oligonucleotide” as used herein refers to a probe that is bound, either covalently, through a linker or a chemical bond, or noncovalently, through ionic, van der Waals, electrostatic, or hydrogen bonds to a label such that the presence of the probe may be detected by detecting the presence of the label bound to the probe.

Nucleic acid probes and primers are readily prepared based on the methods and the nucleic acid sequences disclosed herein. Methods for preparing and using probes and primers and for labeling and guidance in the choice of labels appropriate for various purposes are discussed, e.g., in Sambrook et al., Molecular Cloning, A Laboratory Manual 4th ed. 2012, Cold Spring Harbor Laboratory; and Current Protocols in Molecular Biology, Ausubel et al., eds., 1994, John Wiley & Sons).

The following terms are used to describe the sequence relationships between two or more nucleic acids or polynucleotides: “reference sequence”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length norovirus genome sequence e.g., the C region of the major capsid protein of norovirus, or gene sequence given in a sequence listing.

The terms “identical” or percent “identity,” in the context of two or more nucleic acids or polypeptide sequences, refer to two or more sequences or subsequences that are the same or have a specified percentage of nucleotides that are the same (e.g., 85% identity, 90% identity, 99%, or 100% identity), when compared and aligned for maximum correspondence over a comparison window, or designated region as measured using a sequence comparison algorithm or by manual alignment and visual inspection.

The phrase “substantially identical”, in the context of two nucleic acids or polypeptides, refers to two or more sequences or subsequences that have at least about 85%, identity, at least about 86%, 87%, 88%, 89%, 90%, 91%, 92%, 93%, 94%, 95%, 96%, 97%, 98%, 99%, or 100% nucleotide or amino acid residue identity, when compared and aligned for maximum correspondence, as measured using a sequence comparison algorithm or by visual inspection. In an exemplary embodiment, the substantial identity exists over a region of the sequences that is at least about 50 residues in length. In another exemplary embodiment, the substantial identity exists over a region of the sequences that is at least about 100 residues in length. In still another exemplary embodiment, the substantial identity exists over a region of the sequences that is at least about 150 residues or more, in length. In one exemplary embodiment, the sequences are substantially identical over the entire length of nucleic acid or protein sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

An exemplary algorithm for sequence comparison is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, (1987) J. Mol. Evol. 35:351-360. The method used is similar to the method described by Higgins & Sharp, (1989) CABIOS 5:151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., (1984) Nuc. Acids Res. 12:387-395.

Another example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N4 and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

### I. Introduction:

Noroviruses are non-enveloped, single-stranded RNA viruses in the Caliciviridae family having a genome about 7500 base pairs long. Noroviruses are a leading cause of foodborne illness, and are responsible for over half of the foodborne illness in the world. Noroviruses are grouped by the amino acid sequence of the major capsid protein: viruses with less than 14.3% to 15% difference are classified as strains within the same genotype, 14.3-43.8% difference as genotypes within the same genogroup, and 45-61.4% difference as genogroups within the species norovirus (see e.g., Zheng D. P., et al. (2006) Virology 346: 312-323; MMWR Recomm Rep (2011) 60 No. 3: 1-18). Currently, noroviruses are grouped into five genogroups (GI-GV). Genogroups GI and GII are responsible for most human infections. Genogroups GI and GII are subdivided into 8 and 21 different genotypes, respectively (see e.g., MMWR Recomm Rep (2011) 60 No. 3: 1-18). These genotypes are further subdivided into strains. See e.g., Updated norovirus outbreak management and disease prevention guidelines. MMWR Recomm Rep 60: 1-18).

Five regions of the norovirus genome A, B, C, D and E are commonly used for detection and genotyping (see e.g., Vinjé et al., (2004) J Virol Methods 116(2): 109-117; Mattison et al., (2009) J Clin Microbiol 47(12): 3927-3932). In particular, the norovirus genome regions C and D are considered by the Centers for Disease Control and Prevention (CDC) as the most relevant regions to be used in surveillance studies for identifying a particular norovirus genotype (see e.g., Desai et al., (2012) Clin Infect Dis 55(2): 189-193; Vega et al., (2011) Emerg Infect Dis 17(8): 1389-1395)

The standard method for norovirus detection is quantitative real-time reverse transcription-polymerase chain reaction (RT-PCR)(see e.g., Miura et al. (2013) Appl Environ Microbiol 79(21): 6585-6592; Tong et al., (2011) Water Res 45(18): 5837-5848; Vega et al. (2014) J Clin Microbiol 52(1): 147-155), allowing the detection of some norovirus strains with oligonucleotide probes. Unfortunately however, this approach is limited in the number of probes that can be added in a single reaction due to the interaction between multiple primers and probes. Thus, sensitivity is reduced. Accordingly, the method does not provide sufficient details to distinguish between specific genotypes of norovirus (see e.g., Kageyama et al., (2003) J Clin Microbiol 41(4): 1548-1557; Miura et al. (2013) supra; Verhoef et al., (2010)) Emerg Infect Dis 16(4): 617-624). Typically, definitive identification requires further steps of sequencing the product from RT-PCR requiring additional processing steps for sample analysis (see e.g., Kageyama et al., (2003) J Clin Microbiol 41(4): 1548-1557; Mattison et al., (2011) J Virol Methods 173(2): 233-250; Verhoef et al., (2010) Emerg Infect Dis 16(4): 617-624). Once the sequence of the capsid region, labeled as ORF2 in FIG. 1, has been elucidated the strain type can be identified by determining the closest reference strain match (see e.g., Vega et al. (2011) Emerg Infect Dis 17(8): 1389-1395; Zheng et al., 2006 supra).

Other methods for typing norovirus include DNA microarrays (see e.g., Uttamchandani et al., (2009) Trends Biotechnol 27(1): 53-61). Unfortunately, cross-reactivity is observed using this method when using long probes (larger than 50 nucleotides in length) on the microarray do not discriminate between norovirus strains (see e.g., Brinkman and Fout, (2009) J Virol Methods 156(1-2): 8-18; Mattison et al., (2011) supra).

Thus, what is needed in the art are improved methods for probe design which will permit clear discrimination between norovirus genotypes Fortunately, the disclosure provided herein provides for these and other needs.

### II. Identification of Norovirus and Other Pathogens Using Oligonucleotides

A. General Recombinant DNA Methods

This invention utilizes routine techniques in the field of recombinant genetics. Basic texts disclosing the general methods of use in this invention include Sambrook et al., Molecular Cloning—A Laboratory Manual (2nd Ed.), Vol. 1-3, Cold Spring Harbor Laboratory, Cold Spring Harbor, N. Y., 1989; Kriegler, Gene Transfer and Expression: A Laboratory Manual (1990); and Current Protocols in Molecular Biology (Ausubel et al., eds., 1994)). Unless otherwise noted, technical terms are used according to conventional usage. Definitions of common terms in molecular biology maybe found in e.g., Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

For nucleic acids, sizes are given in either kilobases (kb) or base pairs (bp). Estimates are typically derived from agarose or acrylamide gel electrophoresis, from sequenced nucleic acids, or from published DNA sequences. Oligonucleotides that are not commercially available can be chemically synthesized e.g., according to the solid phase phosphoramidite triester method first described by Beaucage & Caruthers, Tetrahedron Letts. 22:1859-1862 (1981), using an automated synthesizer, as described in Van Devanter et al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Sanger etc 1977 Proc Natl Acad Sci USA. 1977 December; 74(12): 5463-5467

B. Probe Design

The computer program finds probes with a sequence of DNA that is sufficiently complementary to the DNA generated for the targeted genotypes that they will bind to the probe while also being sufficiently uncomplimentary to non-targeted genotypes so those will not bind to the probe.

Probes are designed using a computer program that takes as an input, two files of aligned sequences of nucleotides in the target region in the FASTA file format with the sequence on a single line. The first file contains sequences from strains in the genotype to be detected. The second file contains sequences of other similar genotypes in the same genogroup that should not be detected. The alignment of the sequences can be performed by using any DNA analysis commercial software such as such as Genious (Biomatters Ltd, Auckland, New Zealand). Some sequences may have insertions or deletions relative to the majority of other sequences in the alignment. This is a rare occurrence for sequences in the same genogroup. In such cases, the alignment is made with the insertion or deletion removed from the sequence. After probes are generated, they are verified against the original sequence to validate the expected similarity range. This can be done as part of the post probe design test discussed herein below.

Additionally, the program takes several parameters. The first parameter is the number of oligonucleotides in the desired probe sequence. The second parameter is the minimum percentage by which the probe should match all of the target sequences. The third parameter is the maximum percentage by which the probe should match all non-targeted sequences. The fourth parameter is the number of degenerate oligonucleotides that can be included in the probe sequence.

In one exemplary embodiment, probes were designed to be 25 nucleotides in length, where a minimum of 90% identity for target sequences and a maximum of 79% identity for non-targeted sequences was desired. Starting at the first position of the alignment, the program generates candidate probes of the specified length to match the target. It determines the nucleotide variation at each position of the alignment and generates candidate probe sequences for each variation. Many candidate probes may not match any target sequence at a 100% sequence identity but the candidate probes must meet the specified criteria, which is the minimum sequence identity percentage. The candidate probes are then compared with the non-target sequences to exclude any candidate probes that match a non-target sequence with a percentage greater than the maximum allowable percentage. The program calculates a match score for any probes that pass the filter sequence. The probes in the current invention are 25 nucleotides in length and mismatched all known target genotype strains by no more than 2 nucleotides while having at least 5 mismatches with strains in other genotypes.

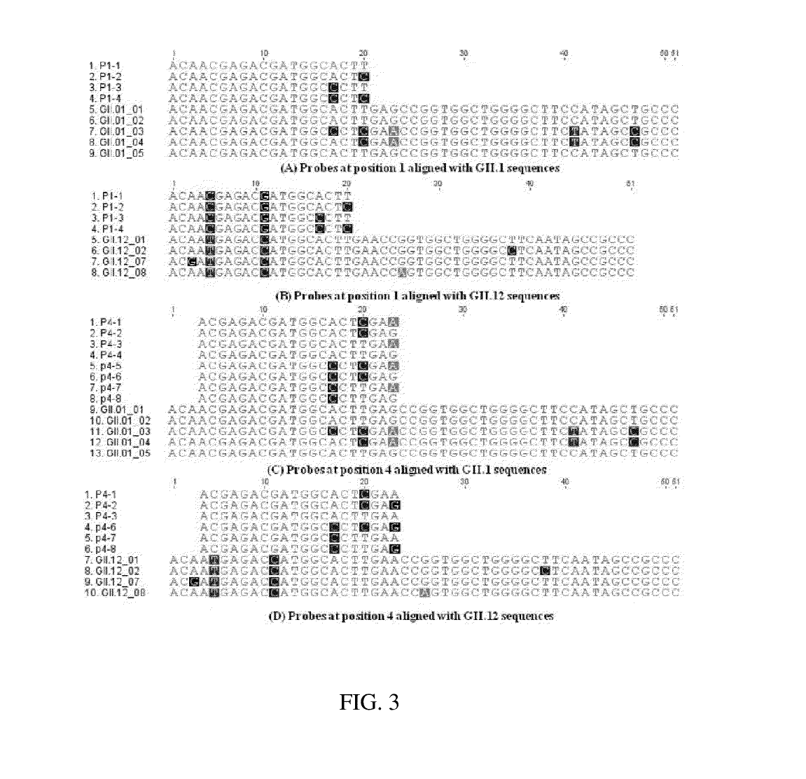

The illustration shown in FIG. 3 shows an exemplary process for generating candidate probes for genotype GII.1 of length 20 that are specific for GII.1 and discriminate against genotype GII.12.

The computer program starts at position 1 and reads all the variants of genotype GII.1 from positions 1-20. Position 17 can have an A or C and position 20 can have a T or C so there are 4 possible probes P1-1, P1-2, P1-3 and P1-4. The probe P1-3 doesn't match any genotype GII.1 sequence at 100% but it matches all of them at least 90% in at least 18 out of 20 nucleotides of all strains; therefore, it is acceptable from the standpoint of being specific for GII.1 (FIG. 3A). While probe P1-4 is 75% identical to the GII.12_{—}07 sequence, that is this probe is identical in 20 of the 25 nucleotides (75%), however it is 80% identical to the other GII.12 sequences so it doesn't sufficiently discriminate against genotype GII.12 sequences (FIG. 3B). The other probes also do not sufficiently discriminate against genotype GII.12 sequences either so they are all discarded. The program then increments its starting position and generates possible probes from positions 2-21. It will find similar results and generate no probes. When the program reaches sequence position 4, it will generate 8 candidate probes with variations of (A and C) at probe position 14, (C and T) at probe position 17 and (A and G) and probe position 20, P4-1, P4-2 to P4-8 (FIG. 3C). Probe P4-4 is not sufficiently selective for sequence GII.01_{—}03 and probe P4-5 is similarly not a sufficient match for sequences GII.01_{—}01, GII.01_{—}02 and GII.01_{—}05, the other probes are sufficiently selective for GII.1 sequences. One probe P4-6 will only be a 75% match with any of the GII.12 sequences and thus also sufficiently discriminating against GII.12 with a discrimination score of 75%. The resulting list of candidate probes are further tested for self-binding characteristics by using primer design software that are commercially available.

C. Reverse Transcription-Polymerase Chain Reaction

Reverse transcription-Polymerase Chain Reaction is well known in the art (see e.g., Shiao YH. (2003) BMC Biotechnol. 2003 Dec. 9; 3:22; J Huggett et al. (2005) Genes and Immunity 6: 279-284).

D. DNA Microarrarys

DNA microarray technology is well known in the art (see e.g., Michael J. Heller (2002) DNA MICROARRAY TECHNOLOGY: Devices, Systems, and Applications, Annual Review of Biomedical Engineering Vol. 4: 129-153; A. Ehrenreich (2006) Applied Microbiology and Biotechnology Vol 73(2): 255-273)

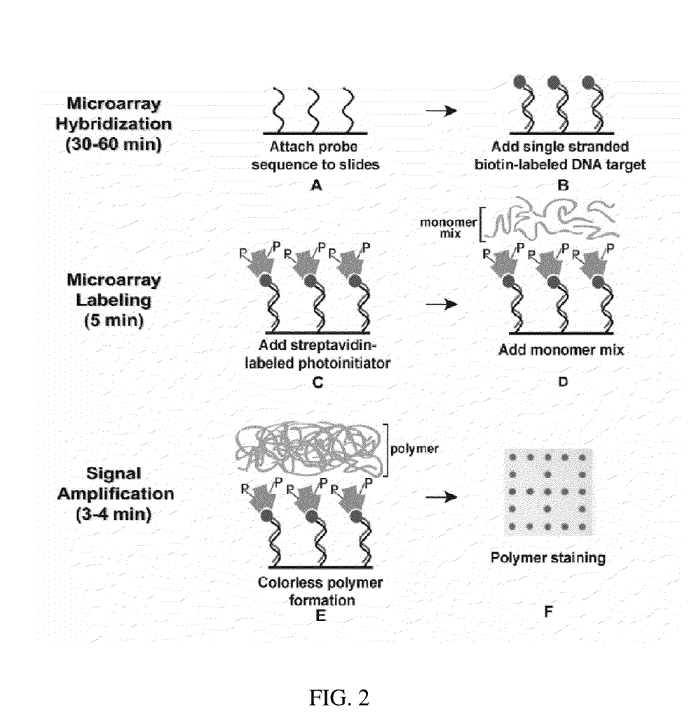

In general, DNA microarray technology comprises the hybridization of a labeled, single stranded test molecule to a specific probe which is attached to a solid support. In an exemplary embodiment, the disclosure provides for the design and synthesis of a specific probe for norovirus GII.4. The specific probe is attached to a solid support for microarray analysis and a sample suspected of comprising a Norovirus GII.4 is labeled and hybridized to the microarray. An exemplary microarray analysis is illustrated in FIG. 2.

### III. Software/Database Structure and Functionality

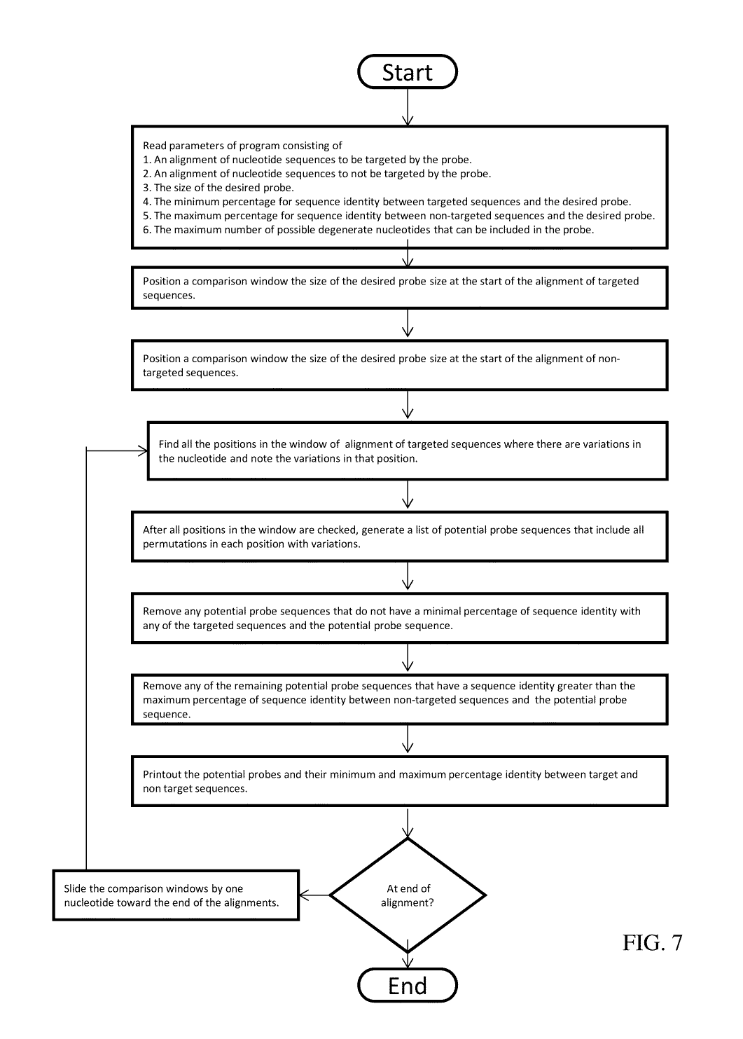

FIG. 7 provides a general overview of the framework for the Probe Design and Identification software. The software is stored on a non-transitory computer readable medium containing computer instructions for causing a computer processor to perform a method for identification of an unknown norovirus strain. The instructions comprising the Probe Design and Identification software are provided herein in the computer program listing appendix.

The following examples are offered to illustrate, but not to limit the invention.

### EXAMPLES

### Example 1

The following example illustrates the design and use of specific norovirus probes for the detection of specific norovirus genotypes such as e.g., GI.2, GI.3a, GI.3b, GI.4, GI.6, GII.1, GII.2 GII.4, and GII.12 by using the norovirus genome region C (Table 1). Additional probes were designed for detecting the strains of genotype GII.4 using the norovirus genome region D (Table 1). The probes were designed by a computer program disclosed in detail herein.

Probes for genotype GII.1 of length 20 that are specific for GII.1 and discriminate against genotype GII.12 were prepared. The program starts at position 1 and reads all the variants of genotype GII.1 from positions 1-20. Position 17 can have an A or C and position 20 can have a T or C so there are 4 possible probes P1-1, P1-2, P1-3 and P1-4. The probe P1-3 doesn't match any genotype GII.1 sequence at 100% but it matches all of them at least 90% i.e. in at least 18 out of 20 nucleotides of all strains; therefore, it is acceptable from the standpoint of being specific for GII.1 (FIG. 3A). While probe P1-4 is less than 79% identical to the GII.12_{—}07 sequence, this probe is identical in 20 of the 25 nucleotides (75%), and it is 80% identical to the other GII.12 sequences so it doesn't sufficiently discriminate against genotype GII.12 sequences (FIG. 3B). The other probes do not sufficiently discriminate against genotype GII.12 sequences either so they are all discarded. The program then increments its starting position and generates possible probes from positions 2-21. It will find similar results and generate no probes. When the program reaches sequence position 4, it will generate 8 candidate probes with variations of (A and C) at probe position 14, (C and T) at probe position 17 and (A and G) and probe position 20, P4-1, P4-2 to P4-8 (FIG. 3C). Probe P4-4 is not sufficiently selective for sequence GII.01_{—}03 and probe P4-5 is similarly not a sufficient match for sequences GII.01_{—}01, GII.01_{—}02 and GII.01_{—}05, the other probes are sufficiently selective for GII.1 sequences. One probe P4-6 will only be a 75% match with the GII.12 sequences which shows 79% identity and thus also sufficiently discriminating against GII.12 and thus an acceptable probe with a discrimination score of 75%. The resulting list of candidate probes are further tested for self-binding characteristics by using primer design software that are commercially available.

### Array Construction

Microarrays were constructed using probes in region C for norovirus strains with genotypes GI.2, GI.3A, GI.3B, GI.4, GI.6, GII.1, GII.2 GII.4, GII.7, and GII.12 were designed as described above. They were purchased from Eurofins Genomics (Huntsville, Ala.) with a 5′-amino-C6 modification for covalent binding to the slide surface. The probes were spotted in duplicate at a final concentration of 50 μMolar on ArrayIt® SuperEpoxy 2 microarray slides (Arrayit Corporation, Sunnyvale, Calif.). As a control for the photopolymerization detection process and marker for the position of genotype specific probes, a synthetic oligonucleotide probe with 5′-amino-C6 and 3′-biotin modifications (InDevR, Inc., Boulder, Colo.) was spotted at a final concentration of 0.5 μMolar. This biotinylated control oligonucleotide did not have any sequence homology to any norovirus genomes. The microarrays were manufactured with an approximate spot diameter size of 200 μm and a center-to-center spacing of 500-700 μm (Arrayit Corporation). After printing, an adhesive microarray well (9 mm diameter, InDevR, Inc.) was placed in the center of the printed array, and the microarray slides were stored in a desiccator until further use, as in previous studies (Quiñones., et al., 2012. Front Cell Inf Microbio 2, 61; Quiñones., et al, 2011. Foodborne Pathog Dis 8, 705-711.).

### Reverse Transcription-Polymerase Chain Reaction

RNA was isolated from human stool samples by methods commonly known to practitioners (see e.g., Tian et al., 2010 J Appl Microbiol 109, 1753-1762) or dilution, filtration and RNA extraction by commercial kit such as QIAamp Viral RNA Extraction Kit (Qiagen, Valencia, Calif.). For norovirus detection using Region C, RT-PCR was performed on the RNA template using G1SKF primer 5′-CTGCCCGAATTYGTAAATGA-3′ (SEQ ID NO:1) and G1SKR primer 5′-CCAACCCARCCATTRTACA-3′ (SEQ ID NO:2) for genogroup I strains (see e.g., Kojima et al., 2002 J Virol Methods 100, 107-114). Region C in genogroup II strains was amplified using G2SKF primer 5′-CNTGGGAGGGCGATCGCAA-3′ (SEQ ID NO:3) and G2SKR primer 5′-CCRCCNGCATRHCCRTTRTACAT-3′(SEQ ID NO:4) (Kojima et al., 2002, supra). For detection using norovirus genome region D in genogroup II strains, RT-PCR was performed on the RNA template using Cap C primer 5′-CCTTYCCAKWTCCCAYGG-3′(SEQ ID NO:5), Cap D3 primer 5′-TGYCTYITICCHCARGAATGG-3′(SEQ ID NO:6, wherein I is deoxyinosine and can base pair with any other nucleotide) and CAP D1 primer 5′-TGTCTRSTCCCCCAGGAATG-3′ (SEQ ID NO:7) (Vinjé et al., 2004 J Virol Methods 116, 109-117). All primers for the RT-PCR step were purchased from Eurofins Genomics (Huntsville, Ala.) with a 5′-phosphorylated modification for the forward primers and a 5′-biotin modification for the reverse primers, as in previous studies (Quiñones et al., 2012; Quiñones et al., 2011, supra). A 50 μl reaction was prepared with 0.6 μMolar of each primer, 10 μl of 5× buffer, 2 μl of dNTP, 2 μl of enzyme from Qiagen® One-Step RT-PCR Kit (Qiagen, Valencia, Calif.) and 1 μl RNA template. The mixture was heated at 50° C. for 30 min then to 95° C. for 15 min in a Dyad Peltier Thermal Cycler (Bio-Rad Laboratories, Hercules, Calif.). PCR cycling conditions for region C amplification consisted of denaturation at 94° C. for 30 sec, primer annealing at 50° C. for 30 sec and extension reaction at 72° C. for 1 min (40 cycles), followed by a final extension at 72° C. for 7 min (Kojima et al., 2002, supra). For region D, the mixture was heated at 42° C. for 60 min then to 95° C. for 15 min (Vinjé et al., 2004, supra). PCR cycling conditions for region D amplification consisted of denaturation at 94° C. for 1 min, primer annealing at 40° C. for 1 min and extension reaction at 72° C. for 1 min (40 cycles), followed by a final extension at 72° C. for 10 min (Vinjé et al., 2004, supra).

### Microarray Hybridization, Labeling and Analysis

For each hybridization reaction, PCR amplicons were purified by using the MinElute® PCR purification kit (Qiagen, Valencia, Calif.). To achieve a rapid microarray hybridization (Boissinot et al., 2007 Clin Chem 53, 2020-2023), single stranded DNA targets were produced after a lambda exonuclease digestion of the PCR-amplified targets with 15 μl of the eluate, 10 U of lambda exonuclease and 1× lambda exonuclease reaction buffer (Epicenter Biotechnologies, Madison, Wis.) in a final volume of 24 μL for 15 min at 37° C., followed by addition of 24 μl of 2× Hybridization Buffer (InDevR, Inc.), as in previous studies (Quiñones et al., 2012, supra; Quiñones et al., 2011, supra). The hybridization mixture was applied to each microarray, and the slides were incubated in a humidified chamber (InDevR, Inc.) for 90 min at room temperature. Following hybridization, the slides were transferred to a slide drying tray (Evergreen Scientific, Los Angeles, Calif.) and were rinsed with Microarray Wash Buffers A thru D (InDevR, Inc.) in the following order: Wash Buffer D for 5 sec, Wash Buffer A for 1 min, Wash Buffer D for 5 sec, Wash Buffer B for 5 min, and Wash Buffer C for 5 min, and then were dried by centrifugation at 200×g for 1 min prior to labeling, as in previous studies (Quiñones et al., 2012, supra; Quiñones et al., 2011, supra).

The hybridized microarrays were labeled after incubation with 40 μl of a streptavidin-conjugated ampliTAG™ labeling solution (InDevR, Inc.) for 5 min at room temperature in a dark humidity chamber, and immediately after labeling, microarrays were rinsed with Wash Buffer D for 5 sec, Wash Buffer C for 5 min, Milli-Q water for 5 min, and then dried by centrifugation at 200×g for 1 min. Positive hybridization signals on each microarray was detected by incubation with 40 μL ampliPHY™ solution (InDevR, Inc.), followed by photoactivation for approximately 1-2 min with the ampliPHOX Reader™ and the associated ampliVIEW™ software 2.1 (InDevR, Inc.), as recommended by the manufacturer. Polymer formation was visualized after a 1 to 2 minute staining with ampliRED™ solution. Color digital images of the stained arrays were acquired with the ampliPHOX Reader, and for each spot, quantification of signal and background mean pixel intensities were determined with the ampliVIEW software (InDevR, Inc.), as in previous studies (Quiñones et al., 2012, supra; Quiñones et al., 2011, supra).

### Results

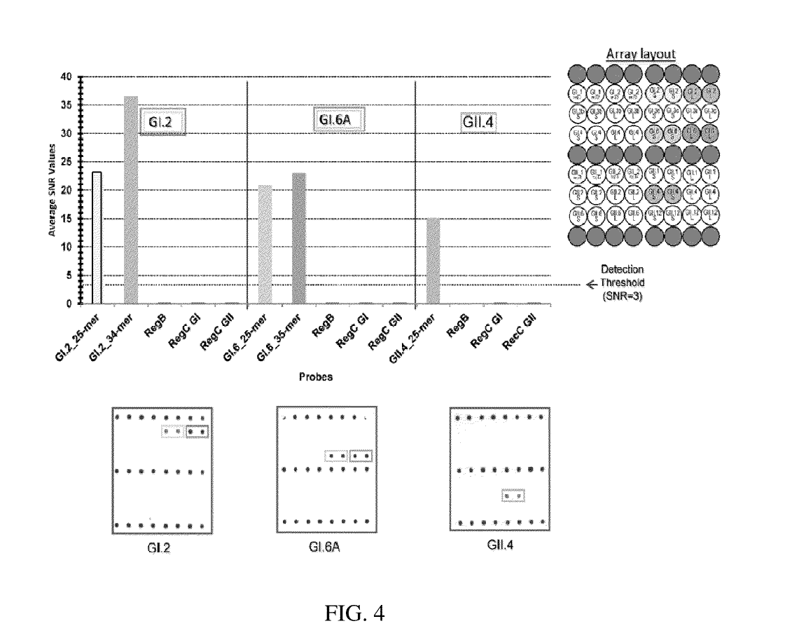

As shown in FIG. 4, the specificity of the probes targeting the norovirus genomic region C for detecting specific genotypes in genogroup I and genogroup II strains was validated. The array layout shows the probes to be tested in different colors, and the spots highlighted in grey represent the controls for the photopolymerization (polymer formation) reaction. The same color scheme, identifying the norovirus-specific probes, was used for the graph. The graph shows the average signal to noise ratio values (SNR) that were quantified for each probe, when testing RNA from various strains with genotypes GI.2, GI.6A, and GII.4. FIG. 4 also shows representative array images when testing RNA from the various norovirus strains, and the results indicated that the patterns of polymer formation correlated with the genotype of the norovirus strains tested. For example, when testing RNA from a GI.2 strain, polymer formed where those probes were spotted on the array (FIG. 4), resulting in high SNR values with an average SNR value of 23.3 (Table 1). By contrast, low SNR values below the detection threshold were observed for the other probes on the array with SNR values of 1.0 or less (FIG. 4 and Table 2). A high level of specificity was also observed when testing RNA from other norovirus strains with this microarray colorimetric method. Furthermore, Table 2 shows the results of probes targeting norovirus region D for identifying GII.4 strains, a genotype that is responsible for >85% of the human infections (Desai et al., 2012 Clin Infect Dis 55, 189-193). High SNR values, ranging from 17.5 to 21.3, were observed for any region D probe when RNA from two representative strains with genotypes GII.4 was tested on the microarray. Finally, no hybridization signals, and consequently, low SNR values was obtained for any region D probes when testing RNA from other strains (Table 2).

TABLE 1
List of 25-mer probes targeting norovirus
genome region C and region D

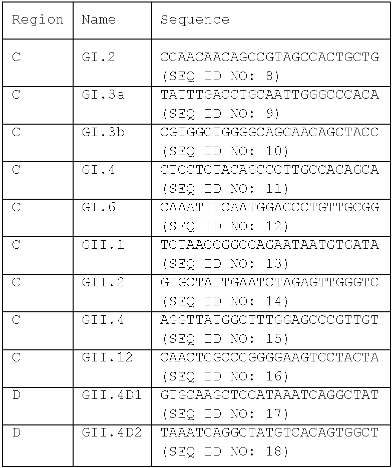

TABLE 2
Signal-to-noise ratio values of 25-mer probes targeting region C and region D when testing norovirus
strains with different genotypes.

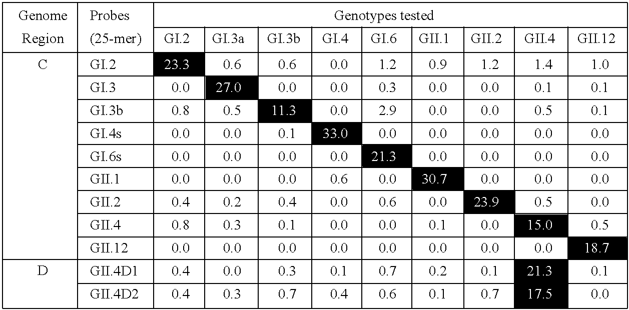

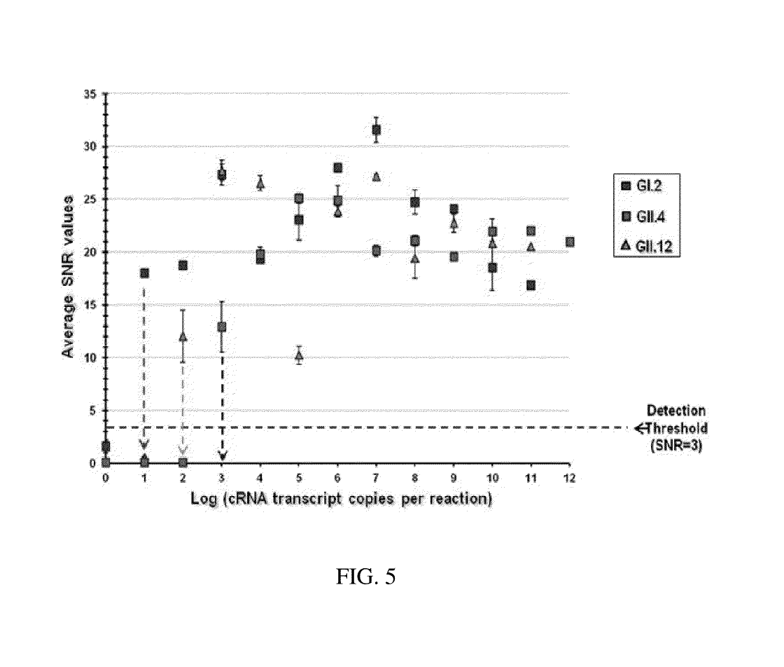

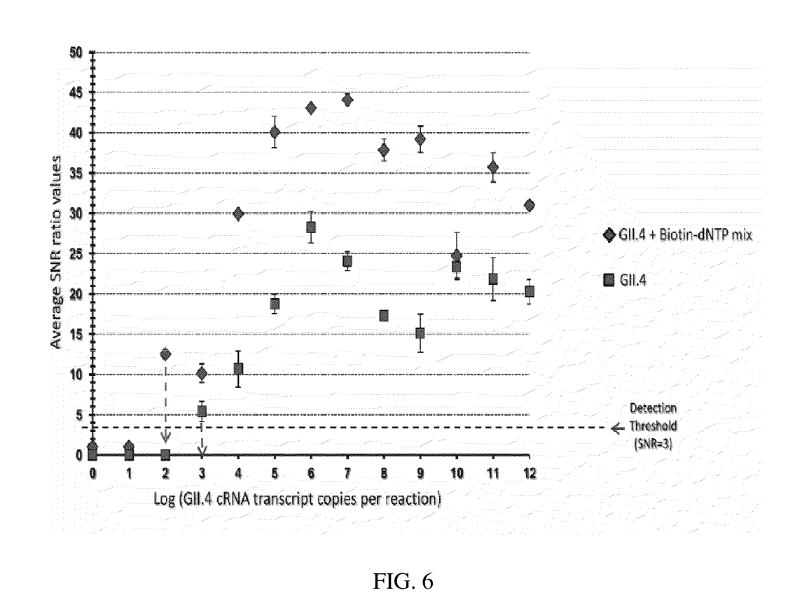

FIG. 5 demonstrates the sensitivity of the probes using the microarray-based colorimetric method. Plasmid DNA controls, targeting region C in norovirus strains with genotypes GI.2, GI.4, GII.4, and GII.12, were constructed to generate cRNA transcripts by using commercially-available, in vitro transcription kits. Results showed that high SNR values were detected when testing higher amounts of the cRNA transcript for all strains tested. When testing lower amounts of the cRNA transcript, high signals were still observed for genotype GI.2 probes with a potential detection limit at 10 transcript copies. A lower sensitivity was observed for detecting genotype GII.12 strains at 100 transcript copies, and an even lower sensitivity was observed for detecting genotype GII.4 strains with a potential detection limit at 1000 transcript copies. To improve the detection sensitivity for genotype GII.4, the amounts of biotin, incorporated in the target during amplification prior to hybridization on the array, were increased (FIG. 6). Biotin is required for polymer formation and for detecting the signals on the array. The results shown in FIG. 6 indicated that by increasing the amounts of biotin in the target allowed us to improve our detection limit to <100 transcript copies per reaction, when compared to our standard method with a detection limit of 1000 transcript copies. This improved detection sensitivity to about 100 transcript copies is within the range documented by other standard methods such as real time reverse transcription-PCR.

### Example 2

The following Example illustrates use of the methods disclosed herein for the design of specific probes useful for distinguishing hepatitis strains. In particular, the following example illustrates an exemplary design of a pair of probes to detect an important hepatitis A genotype IB. This genotype was involved in an important multistate outbreak of hepatitis A. See Collier M. G., et al., (2014) The Lancet Infectious Diseases Volume 14, Issue 10, October 2014, Pages 976-981

A total of 65 different hepatitis A virus genomes are downloaded from the NCBI (National Center for Biotechnology Information) database. The sequence of the hepatitis AVP1-2B genome region, a junction region at the end of the capsid region and beginning of the non-structural proteins and used by the CDC to genotype hepatitis A, is extracted from each genome, and all the sequences are then aligned. The 15 sequences from the genotype IB genomes are used as the in silico genomic data of the target subtype. The remaining 50 sequences are the in silico genomic data of the non-target subtype.

Probes are designed according to the method disclosed herein. The sequence identity for the target subtype is at least 90% (low variability) and the sequence identity for the non-target subtype is no more than 75% (high variability). As an example, two probes will detect hepatitis 1B genotype. In particular, the in silico 15 genome sequences representing genotype IB can be divided into 2 groups: the first group contains 7 genome sequences and the second group contains 8 genome sequences. By using the method of claim 1, probes will be designed with the 7 genome sequences of the first group and the 8 sequences of the second group as the in silico genomic data of the target species. The probe pairs, one probe for the first group and one probe for the second group, will distinguish hepatitis A genotype IB from other hepatitis A genotypes.

### Example 3

The following Example illustrates use of the methods disclosed herein for the design of specific probes useful for distinguishing pathogenic Escherichia coli strains. In particular, the following example illustrates an exemplary design of specific probes to detect an important genotype of pathogenic Escherichia coli that produce Shiga toxins, which have been implicated in causing severe human illness. See http://www.cdc.gov/ecoli/general/.

Different genomes of Escherichia coli strains representing different 0-antigen groups are downloaded from the NCBI (National Center for Biotechnology Information) database. The sequences of the different virulence genes encoding the toxin molecules such as the Shiga toxin subtypes (stx genes) as well as the adhesion molecules such as the intimin subtypes (eae genes), which are used to genotype Escherichia coli.

Probes can be designed according to the method disclosed herein. The sequence identity for the target subtype would be at least 90% (low variability) and the sequence identity for the non-target subtype would be no more than 75% (high variability) for all probes detecting toxin or adhesion molecules.

It is understood that the examples and embodiments described herein are for illustrative purposes only and that various modifications or changes in light thereof will be suggested to persons skilled in the art and are to be included within the spirit and purview of this application and scope of the appended claims.

What is claimed is:

1. A method designing a probe specific for a target subtype, wherein the probe specific for the target subtype distinguishes between the target subtype and all known non-target subtypes, the method comprising:
(i) providing in silico genomic data comprising an alignment of nucleotide sequences that comprise the target subtype,
(ii) sequentially scanning the in silico genomic data comprising the alignment of nucleotide sequences that comprise the target subtype, using a select comparison window, to determine a region of low variability between the nucleotide sequences that comprise the target subtype, thereby identifying one or more potential probes, each of which are a length in nucleotides equal to the select comparison window;
(iii) providing an in silico alignment of nucleotide sequences of each of the known non-target subtypes,
(iv) testing the one or more potential probes against the in silico alignment of nucleotide sequences of each of the non-target subtypes to determine if any of the potential probes have high variability with the nucleotide sequences of the in silico alignment of nucleotide sequences of each of the non-target subtypes, thereby identifying one or more specific probes;
(v) selecting the specific probes that show low variability to the nucleotide sequence of the target subtype and high variability to in silico alignment of nucleotide sequences of each of the non-target subtypes,
thereby
designing the specific probes for the target subtype.

2. The method of claim 1, wherein the low variability is at least 90% sequence identity and wherein the low variability is less than 79%.

3. The method of claim 1, wherein the target subtype is a member selected from the group consisting of a norovirus genotype, a strain of hepatitis A virus and a strain of Escherichia coli.

4. A method for designing a probe specific for a target norovirus genotype, wherein the probe specific for the target norovirus genotype distinguishes between the target norovirus genotype and all known non-target norovirus genotypes, the method comprising:
(i) providing in silico genomic data comprising an alignment of nucleotide sequences from the target norovirus genotype,
(ii) sequentially scanning the in silico genomic data comprising the alignment of nucleotide sequences from the target norovirus genotype-using a select comparison window to determine a region of low variability between the nucleotide sequences of the target norovirus genotype, thereby identifying one or more potential probes, each of which are a length in nucleotides equal to the select comparison window;
(iii) providing an in silico alignment of nucleotide sequences of each of the known non-target norovirus genotypes,
(iv) testing the one or more potential probes against the in silico alignment of nucleotide sequences of each of the non-target norovirus genotypes to determine if any of the potential probes have high variability with the nucleotide sequences of the in silico alignment of nucleotide sequences of each of the non-target norovirus genotypes, thereby identifying specific probes;
(v) selecting the specific probes that show low variability to the alignment of nucleotide sequences from the target norovirus genotype and high variability to in silico alignment of nucleotide sequences of each of the non-target norovirus genotypes,
thereby
designing the specific probes for the target norovirus genotype.

5. The method of claim 4, wherein the low variability is at least 90% sequence identity and the high variability is less than 75% sequence identity.

6. The method of claim 4, wherein the select comparison window is between about 20 nucleotides to about 40 nucleotides.

7. A non-transitory computer readable medium containing computer instructions stored therein for causing a computer processor to perform a method for designing a probe specific for a target norovirus wherein the method for designing the probe specific for a target norovirus strain comprises:
(i) providing in silico genomic data comprising an alignment of nucleotide sequences from the target norovirus genotype,
(ii) sequentially scanning the in silico genomic data comprising the alignment of nucleotide sequences from the target norovirus genotype-using a select comparison window to determine a region of low variability between the nucleotide sequences of the target norovirus genotype, thereby identifying one or more potential probes, each of which are a length in nucleotides equal to the select comparison window;
(iii) providing an in silico alignment of nucleotide sequences of each of the known non-target norovirus genotypes,
(iv) testing the one or more potential probes against the in silico alignment of nucleotide sequences of each of the non-target norovirus genotypes to determine if any of the potential probes have high variability with the nucleotide sequences of the in silico alignment of nucleotide sequences of each of the non-target norovirus genotypes, thereby identifying specific probes;
(v) selecting the specific probes that show low variability to the alignment of nucleotide sequences from the target norovirus genotype and high variability to in silico alignment of nucleotide sequences of each of the non-target norovirus genotypes,
thereby
designing the specific probes for the target norovirus genotype.

8. A method for determining if a target norovirus genotype is present in a sample comprising a population of noroviruses, the method comprising:
(i) designing a probe specific for the target norovirus wherein designing the probe specific for the target norovirus comprises:
(a) providing in silico genomic data comprising an alignment of nucleotide sequences from the target norovirus genotype,
(b) sequentially scanning the in silico genomic data comprising the alignment of nucleotide sequences from the target norovirus genotype using a select comparison window to determine a region of low variability between the nucleotide sequences of the target norovirus genotype, thereby identifying one or more potential probes, each of which are a length in nucleotides equal to the select comparison window;
(c) providing an in silico alignment of nucleotide sequences of each of the known non-target norovirus genotypes,
(d) testing the one or more potential probes against the in silico alignment of nucleotide sequences of each of the non-target norovirus genotypes to determine if any of the potential probes have high variability with the nucleotide sequences of the in silico alignment of nucleotide sequences of each of the non-target norovirus genotypes, thereby identifying specific probes;
(e) selecting the specific probes that show low variability to the alignment of nucleotide sequences from the target norovirus genotype and high variability to in silico alignment of nucleotide sequences of each of the non-target norovirus genotypes,
thereby
designing the specific probes for the target norovirus genotype;
(ii) attaching the specific probes for the target norovirus to a microarray slide;
(iii) preparing labeled DNA from a sample comprising target norovirus nucleotide sequences and non-target norovirus nucleotide sequences so that the labeled cDNA comprises labeled target norovirus cDNA and labeled non-target norovirus cDNA;
(iv) hybridizing the labeled cDNA comprising labeled target norovirus cDNA and labeled non-target norovirus cDNA to the microarray slide to which the specific probes for the target norovirus strain is attached;
(v) detecting a hybridization signal when the labeled target norovirus cDNA specifically hybridizes to the specific probe for the target norovirus attached to the microarray slide,
thereby determining if a target norovirus is present in a sample comprising a population of noroviruses.


SEQUENCE LISTING (18 sequences)

SEQ ID NO: 1 - DNA, 20 bp, Artificial Sequence
  PRIMER
ctgcccgaattygtaaatga

SEQ ID NO: 2 - DNA, 19 bp, Artificial Sequence
  PRIMER
ccaacccarccattrtaca

SEQ ID NO: 3 - DNA, 19 bp, Artificial Sequence
  PRIMER
cntgggagggcgatcgcaa

SEQ ID NO: 4 - DNA, 23 bp, Artificial Sequence
  PRIMER
ccrccngcatrhccrttrtacat

SEQ ID NO: 5 - DNA, 18 bp, Artificial Sequence
  PRIMER
ccttyccakwtcccaygg

SEQ ID NO: 6 - DNA, 21 bp, Artificial Sequence
  PRIMER
tgyctyntncchcargaatgg

SEQ ID NO: 7 - DNA, 20 bp, Artificial Sequence
  PRIMER
tgtctrstcccccaggaatg

SEQ ID NO: 8 - DNA, 25 bp, Artificial Sequence
  PRIMER
ccaacaacagccgtagccactgctg

SEQ ID NO: 9 - DNA, 25 bp, Artificial Sequence
  PRIMER
tatttgacctgcaattgggcccaca

SEQ ID NO: 10 - DNA, 25 bp, Artificial Sequence
  PRIMER
cgtggctggggcagcaacagctacc

SEQ ID NO: 11 - DNA, 25 bp, Artificial Sequence
  PRIMER
ctcctctacagcccttgccacagca

SEQ ID NO: 12 - DNA, 25 bp, Artificial Sequence
  PRIMER
caaatttcaatggaccctgttgcgg

SEQ ID NO: 13 - DNA, 25 bp, Artificial Sequence
  PRIMER
tctaaccggccagaataatgtgata

SEQ ID NO: 14 - DNA, 25 bp, Artificial Sequence
  PRIMER
gtgctattgaatctagagttgggtc

SEQ ID NO: 15 - DNA, 25 bp, Artificial Sequence
  PRIMER
aggttatggctttggagcccgttgt

SEQ ID NO: 16 - DNA, 25 bp, Artificial Sequence
  PRIMER
caactcgcccggggaagtcctacta

SEQ ID NO: 17 - DNA, 25 bp, Artificial Sequence
  PRIMER
gtgcaagctccataaatcaggctat

SEQ ID NO: 18 - DNA, 25 bp, Artificial Sequence
  PRIMER
taaatcaggctatgtcacagtggct